(19) United States
(12) Patent Application Publication
(10) Pub. No.: US 2011/0124671 A1
(43) Pub. Date: May 26, 2011

(54) SPIROINDENES AND SPIROINDANES AS MODULATORS OF CHEMOKINE RECEPTORS

(76) Inventors: Brian W. Budzik, King of Prussia, PA (US); Michael Jonathan Bury, King of Prussia, PA (US); Minghua Gu, Collegeville, PA (US); Ronggang Liu, King of Prussia, PA (US); Feng Ren, Shanghai (CN); Clark A. Sehon, King of Prussia, PA (US); Gren Z. Wang, King of Prussia, PA (US); Jing Zhang, King of Prussia, PA (US)

(21) Appl. No.: 12/673,172

(22) PCT Filed: Aug. 14, 2008
(86) PCT No.: PCT/US08/73099

§ 371 Date: Feb. 12, 2010

Related U.S. Application Data
(60) Provisional application No. 60/955,675, filed on Aug. 14, 2007.

(51) Int. Cl. A61K 31/438; C07D 401/06; A61P 25/28; A61P 9/10; A61P 29/00; A61P 31/16; A61P 3/00; A61P 11/06; A61P 13/12; A61P 1/00; A61P 3/10

(52) U.S. Cl. 514/278; 546/17

(57) ABSTRACT

The present invention relates to a compound of the following formula:

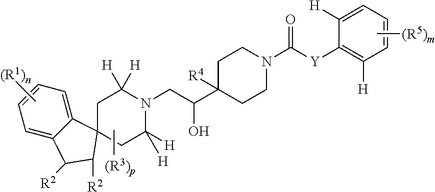

or a pharmaceutically acceptable salt thereof;
where R¹-R⁵, Y, m, n, and p are defined herein. Compounds and compositions of the present invention are useful the treatment of atherosclerosis.

The present invention relates to a class of spiroindenes and spiroindanes that are modulators of chemokine receptors, particularly as CCR2 antagonists and their methods of use.

CCR2 is a chemokine receptor that is expressed on a cell surface of monocycles and some other blood leukocytes. CCR2 binds to the monocyte chemotactic protein MCP-1, and other CC chemokines, which are produced at sites of inflammation and infection. Recruitment of monocytes to inflammatory sites by MCP-1/CCR2 interactions has been implicated in driving the pathogenesis of a number of diseases including multiple inflammatory disorders including rheumatoid arthritis, atherosclerosis, multiple sclerosis, bronchiolitis obliterans syndrome, asthma, allergic rhinitis, eczema, atopic dermatitis, kidney disease, alveolitis, nephritis, liver cirrhosis, congestive heart failure, viral meningitis, cerebral infarction, neuropathy, Kawasaki disease, Alzheimer's disease, stroke, acute nerve injury, HIV infection, AIDS, autoimmune diseases, cancer, sepsis, retinosis, inflammatory bowel disease, transplant arteriosclerosis, idiopathic pulmonary fibrosis, psoriasis, HIV-associated dementia, lupus, erthematosis, hepatitis, pancreatitis, Crohn's disease, endometriosis, and diabetes.

Accordingly, it would be an advance in the art to discover a class of compounds that bind to CCR2, thereby preventing or minimizing the formation of the undesirable MCP1-mediated recruitment of monocytes to inflammatory sites.

### SUMMARY OF THE INVENTION

In a first aspect, the present invention is a compound of formula I represented by the following structure:

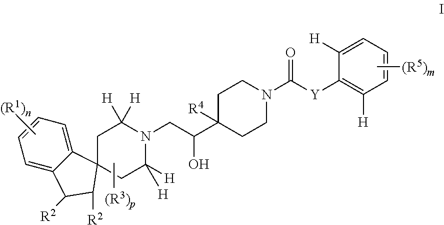

or a pharmaceutically acceptable salt thereof;
where each R¹ is independently halo, CF₃, C₁-C₄-alkyl, C₁-C₄-alkoxy, OCF₃, CN, C₁-C₆-alkyl-C(O)—NH—, C₁-C₆-alkyl-NH—C(O)—, —CH₂—N(R⁶)₂, —CH₂—O—R⁷, or heteroaryl;
each R² is H or, together with carbon atoms to which they are attached, form a double bond;
each R³ is each independently C₁-C₄-alkyl, hydroxy-C₁-C₄-alkyl, or C₁-C₄-alkoxy;
R⁴ is H, OH, F, CN, CF₃, or C₁-C₆-alkyl;
each R⁵ is independently halo, CF₃, C₁-C₄-alkyl, C₁-C₄-alkoxy, OCF₃, benzyloxy, or CN;
each R⁶ is independently H, C₁-C₄-alkyl, or, together with the nitrogen atom to which they are attached, form a 5- or 6-membered heterocycloalkyl group;
R⁷ is H, C₁-C₆-alkyl, benzyl, or phenyl;

### Y is —NH— or

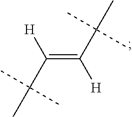

n is 0, 1, or 2;
m is 0, 1, 2 or 3; and
p is 0, 1, or 2.

In a second aspect, the present invention relates to a composition comprising the compound of Formula I, or a pharmaceutically acceptable salt thereof, and a pharmaceutically acceptable excipient.

In a third aspect, the present invention is a method of treating atherosclerosis comprising administering to a patient in need thereof a pharmaceutically effective amount of the compound of Formula I or a pharmaceutically acceptable salt thereof.

Compounds and compositions of the present invention are useful for the treatment of atherosclerosis.

### DETAILED DESCRIPTION OF THE INVENTION

In a first aspect, the present invention relates to a compound of the following formula:

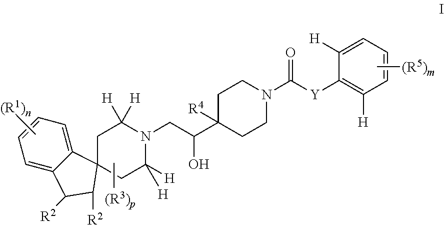

or a pharmaceutically acceptable salt thereof, wherein R¹-R⁵, n, m, and p are as previously defined.

As used herein, C₁-C₆-alkyl and C₁-C₄-alkyl refer to straight or branched hydrocarbon chains containing the specified number of carbon atoms. Examples include methyl, ethyl, n-propyl, n-butyl, isobutyl, isopropyl, t-butyl, and 1,1-dimethylpropyl.

Examples of C₁-C₄-alkoxy include methoxy, ethoxy, n-propoxy, prop-2-oxy, n-butoxy, but-2-oxy, 2-methylprop-1-oxy, and 2-methylprop-2-oxy.

Examples of hydroxy-C₁-C₆-alkyl include hydroxymethyl, 2-hydroxyethyl, 1-hydroxyethyl, 3-hydroxypropyl, 2-hydroxypropyl, and 1-hydroxypropyl.

R⁸ and R⁹, together with the nitrogen atom to which they are attached, may form a 5-6-membered heterocycloaliphatic ring, examples of which include pyrrolidinyl, morpholino, thiomorpholino, dihydropyridazinyl, piperidinyl, piperazinyl, and 4-methylpiperazinyl.

As used herein, heteroaryl refers to a 5- or 6-membered aromatic group that contains one or more heteroatoms selected from N, S, and O. Examples of heteroaryl groups include pyrrolyl, furyl, thienyl, pyrazolyl, imidazolyl, pyridinyl, pyridazinyl, pyrimidinyl, oxazolyl, thiazolyl, isoxazolyl, and isothiazolyl.

As used herein, “halo” refers to fluoro, chloro, or bromo.

As used herein, the term “a compound” or “the compound” refers to one or more compounds of the present invention. Compounds of the present invention may exist in solid or liquid form. In the solid state, they may exist in crystalline or noncrystalline form, or as a mixture thereof. The skilled artisan will appreciate that pharmaceutically acceptable solvates may be formed for crystalline compounds wherein solvent molecules are incorporated into the crystalline lattice during crystallization. Solvates include water, as well as non-aqueous solvents such as ethanol, isopropanol, DMSO, acetic acid, ethanolamine, and ethyl acetate incorporated into the crystalline lattice. Solvates with water incorporated into the crystalline lattice are typically referred to as “hydrates.” Hydrates include stoichiometric hydrates as well as compositions containing variable amounts of water. The invention includes all such solvates.

The present invention includes compounds as well as their pharmaceutically acceptable salts. Accordingly, the word “or” in the context of “a compound or a pharmaceutically acceptable salt thereof” is understood to refer to either a compound or a pharmaceutically acceptable salt thereof (alternative), or a compound and a pharmaceutically acceptable salt thereof (in combination).

As used herein, the term “pharmaceutically acceptable” refers to those compounds, materials, compositions, and dosage forms which are, within the scope of sound medical judgment, suitable for use in contact with the tissues of human beings and animals without excessive toxicity, irritation, or other problem or complication, commensurate with a reasonable benefit/risk ratio. The skilled artisan will appreciate that pharmaceutically acceptable salts of compounds according to formula (I) may be prepared. These pharmaceutically acceptable salts may be prepared in situ during the final isolation and purification of the compound, or by separately reacting the purified compound in its free acid or free base form with a suitable base or acid, respectively.

Compounds of the present invention can form pharmaceutically acceptable salts by reaction with a suitable inorganic or organic acid; examples of suitable inorganic acids include hydrochloric, hydrobromic, phosphoric, metaphosphoric, nitric, and sulfuric acids; examples of suitable organic acids include tartaric, acetic, trifluoroacetic, citric, malic, lactic, fumaric, benzoic, formic, propionic, glycolic, gluconic, maleic, succinic, methanesulfonic, ethanesulfonic, stearic, benzenesulfonic, bromobenzenesulfonic, and p-toluenesulfonic acids.

Compounds of the present invention may exist in stereoisomeric forms. For example, compounds of the present invention contain a hydroxyethylene linker between piperidinyl groups that may be prepared as a racemic mixture or as individual enantiomers. Moreover, when R³ is a substituent such as methyl, an additional two asymmetric centers are introduced into the molecule, as illustrated:

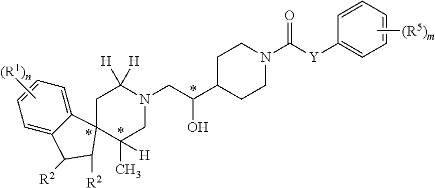

The two additional asymmetric centers are also manifest, for example, when the 3′ position of the spiro-piperidine group is disubstituted or the 3′ and 5′ positions are substituted.

The enantiomers may be prepared, for example, using chiral reagents or separated by chromatography using a chiral column or, if necessary, resolved using a suitable agent such as (S,S)-Co(Salen) or (R,R)-Co(Salen). The individual stereoisomers and mixtures thereof are included within the scope of the present invention.

In a further embodiment, the present invention is a compound of Formula I or a pharmaceutically acceptable salt thereof, wherein m is 1 or 2 and R⁵ is F, Cl, Br, —OCH₃, —CH₃, OCF₃, or O-benzyl.

In a further embodiment of the present invention, p is 0 or 1 and R³ is CH₃.

In a further embodiment, the present invention is a compound of Formula II or a pharmaceutically acceptable salt thereof:

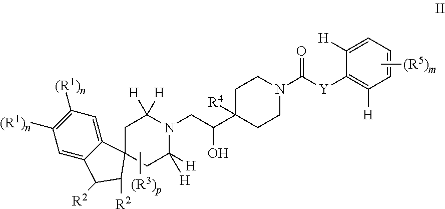

where each n is independently 0 or 1.

In a further embodiment, the present invention is a compound of Formula III, or a pharmaceutically acceptable salt thereof:

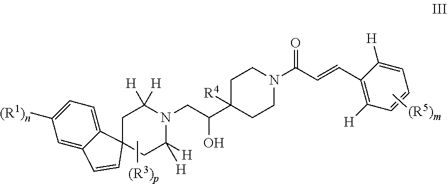

where n is 0 or 1; and R¹ is CH₃, F, or Cl.

In a further embodiment of the present invention each R² is H; and Y is —NH—.

In a further embodiment of the present invention each R² is H; and Y is

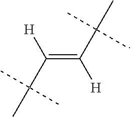

In a further embodiment, the present invention is a compound of Formula I, or a pharmaceutically acceptable salt thereof, which compound is selected from the group consisting of:
1-{1-[(2E)-3-(3,5-difluorophenyl)-2-propenoyl]-4-piperidinyl}-2-(1′H-spiro[indene-1,4′-piperidin]-1′-yl)ethanol;
2-(5-chloro-1′H-spiro[indene-1,4′-piperidin]-1′-yl)-1-{1-[(2E)-3-(3,5-difluorophenyl)-2-propenyl]-4-piperidinyl}ethanol
1-{1-[(2E)-3-(3,5-difluorophenyl)-2-propenoyl]-4-piperidinyl}-2-[(1R,3′R)-3′-methyl-1′H-spiro[indene-1,4′-piperidin]-1′-yl]ethanol; and
1-{1-[(2E)-3-(3,5-difluorophenyl)-2-propenoyl]-4-piperidinyl}-2-[(1S,3′S)-3′-methyl-1′H-spiro[indene-1,4′-piperidin]-1′-yl]ethanol.

In a further embodiment, the invention provides method of treating a disease comprising administering the compound or composition of the present invention or a pharmaceutically acceptable salt thereof to a patient in need thereof, wherein the disease is atherosclerosis, inflammatory pain, influenza, metabolic syndrome, multiple sclerosis, asthma, kidney disease, congestive heart failure, Alzheimer's disease, stroke, Crohn's disease, inflammatory bowel disease, endometriosis, or diabetes.

While it is possible that a compound of the present invention may be administered as the pure chemical, it is generally preferable to administer the active ingredient as a pharmaceutical formulation. Accordingly, in a further aspect, the invention provides a pharmaceutical composition comprising a compound of formula T or a pharmaceutically acceptable salt thereof together with one or more pharmaceutically acceptable carriers or diluents. The carrier(s), diluent(s) and/or excipient(s) must be acceptable in the sense of being compatible with the other ingredients of the formulation and not deleterious to the patient.

Compounds of the invention may be administered in conventional dosage forms prepared by combining a compound of the invention with standard pharmaceutical carriers, diluents or excipients according to conventional procedures well known in the art. These procedures may involve mixing, granulating and compressing or dissolving the ingredients as appropriate to the desired preparation.

The compositions may be in the form of tablets, capsules, powders, granules, lozenges, creams or liquid preparations, such as oral or sterile parenteral solutions or suspensions.

Tablets and capsules for oral administration may contain conventional excipients including binding agents, fillers, lubricants, disintegrants, and wetting agents such as those well known in the art. The tablets may be coated according to methods well known in the art.

### Affinity for CCR2 Receptor

Compounds of the present invention have been found to exhibit affinity for chemokine receptors, in particular the CCR2 receptor. Such affinity is typically calculated from the IC₅₀ as the concentration of a compound necessary to inhibit 50% of the stimulated response from the receptor in an appropriate assay, and is reported as a “Kᵢ” value calculated by the following equation:

$${K}_{i} = \frac{{IC}_{50}}{1 + L / {K}_{D}}$$

where L=radioligand and K_{D}=affinity of radioligand for receptor (Cheng and Prusoff, Biochem. Pharmacol. 22:3099, 1973).

In the context of the present invention pKi (corresponding to the antilogarithm of Ki) is used instead of Ki.

CCR-2 [³⁵S] GTPgS SPA binding assay

### Membrane Preparation

CHO cells expressing the human CCR-2 receptor were grown in DMEM F12 media supplemented with 10% foetal calf scrum, 2 mM L-glutamine, G418 at 37° C. 5% CO₂. Confluent cells were harvested using Hanks buffered salt solution (HBSS, Ca²⁺, Mg²⁺free) containing 0.6 mM EDTA. The resulting cell suspension was centrifuged at 300 g at 4° C. for 10 min, cell pellet resuspended in 100 mL HBSS+EDTA and respun at 300 g for 5 min. The resulting cell pellet was resuspended in 50 mM HEPES containing 100 mM leupeptin, 25 μg/mL bacitracin, 1 mM EDTA, 1 mM PMSF and 2 μM pepstain A, at pH 7.4. The suspension was homogenised using an ice cold blender and centrifuged at 500 g for 20 min. The supernatant is withdrawn and spun at 48000 g for 30 min. This cell pellet is resuspended in the above buffer minus the pepstatin A and PMSF and stored in aliquots at −70° C.

### Assay

For the assay, membranes were thawed and resuspended in assay buffer (20 mM HEPES, 10 mM MgCl₂, 100 mM NaCl, pH 7.4, containing 1 mg/mL saponin, 10 mM GDP) to give a final concentration of 5 μg/well. The membranes were pre-coupled with LEADseeker SPA beads (0.25 mg/well) for 30 min at room temperature while mixing. Assay plates containing 0.5 μL of various test compounds (30 μM-30 μM) in 100% DMSO as 11 point, four fold dilutions across a 384 well plate were used in the assay which have been prepared on a Biomek FX. The plate also contained 16 wells of DMSO and 16 wells of a high concentration of a standard antagonist to produce high and low controls in the experiment. To this 15 μL of bead and membrane mix were added with, 15 μL [³⁵S] GTPgS (0.2 nM final assay concentration) and 15 μL of an EC₈₀ (40 nM) of MCP-1. This concentration of MCP-1 had been pre-determined from agonist curves run against this receptor. All additions were made using a multidrop. Plates were then sealed and centrifuged for 5 min at 300 rpm before they were left to incubate at room temperature for 3 h. After this time they were read on a Viewlux imaging system. For data handling the high and low controls wells were used to normalize the data, which was then fitted using a 4 parameter kit in Excel.

The assay described above is believed to have an effective limit of detection of a pKi in the region of 5.0-5.5. Accordingly, a compound exhibiting a pKi value within this range from such an assay may indeed have a reasonable affinity for the receptor, but equally it may also have a lower affinity, including a considerably lower affinity. Using this assay, all of the exemplified compounds gave a of pKi≧6.

### Schemes

The following schemes illustrate how compounds of the present invention can be prepared. The specific solvents and reaction conditions referred to are also illustrative and are not intended to be limiting. Compounds not described are either commercially available or are readily prepared by one skilled in the art using available starting materials.

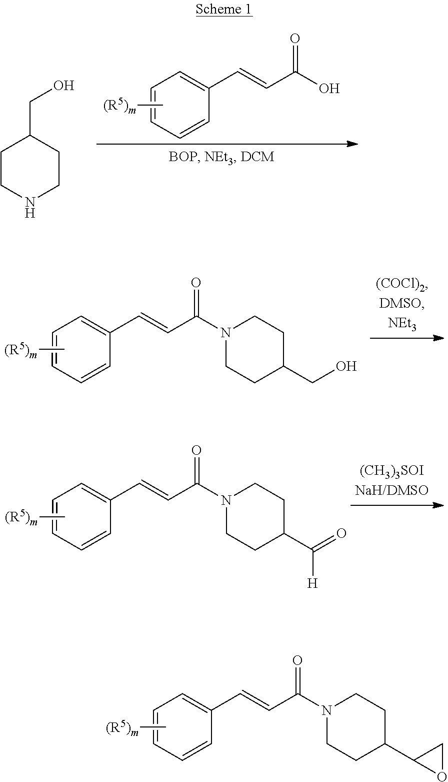

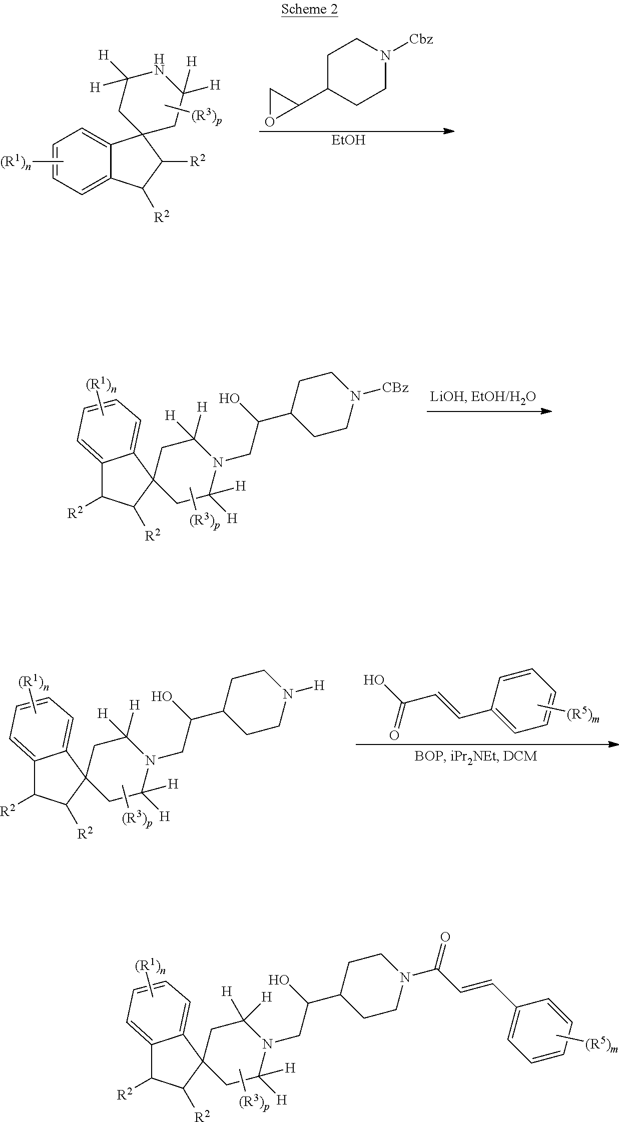

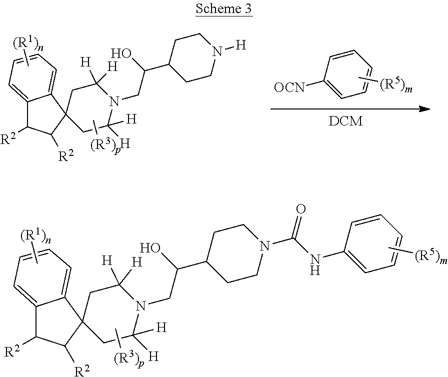

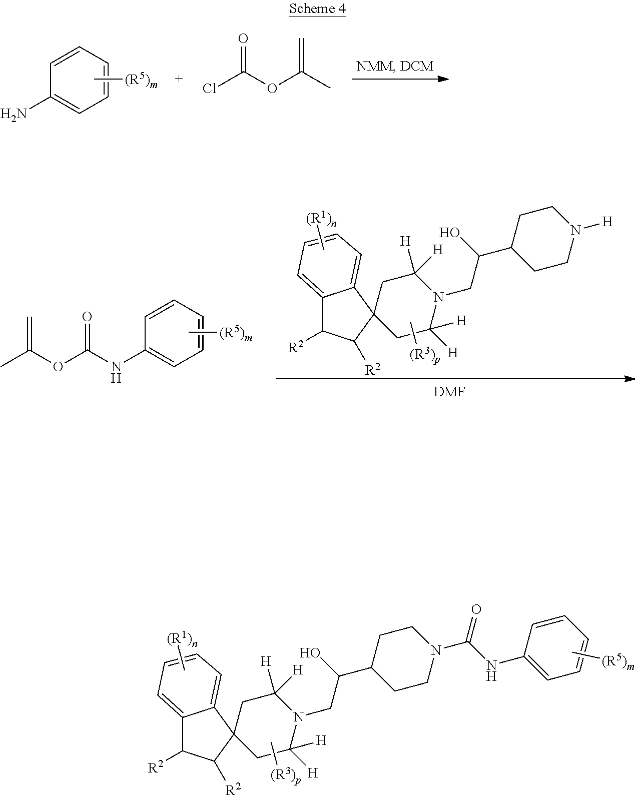

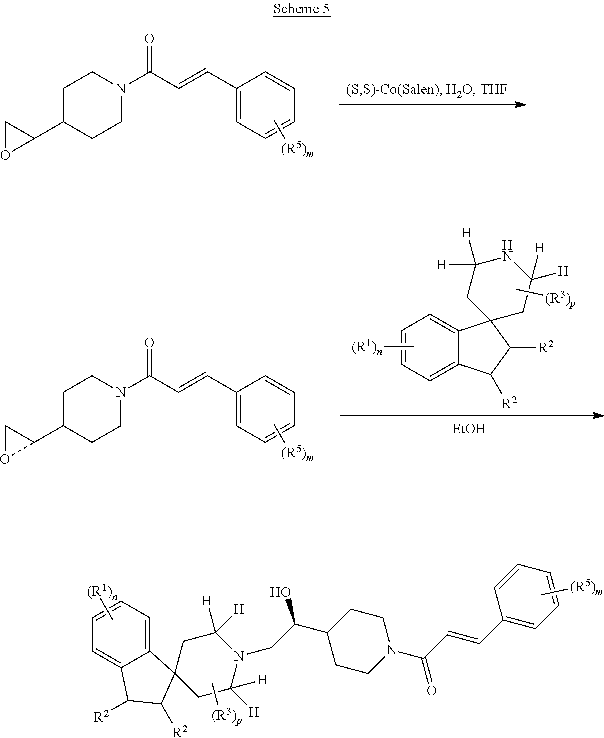

The racemate 4-(2-oxiranyl)-1-[(2E)-3-phenyl-2-propenoyl]piperidine can also be reacted with (R,R)-Co(Salen) to form 4-[(2R)-2-oxiranyl]-1-[(2E)-3-phenyl-2-propenoyl]piperidine

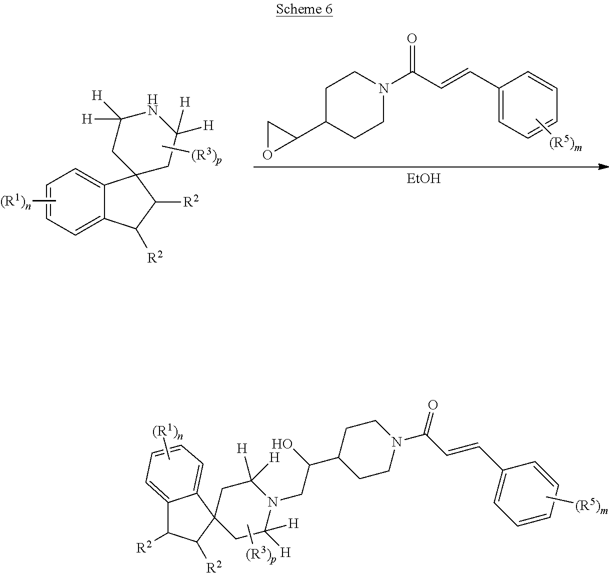

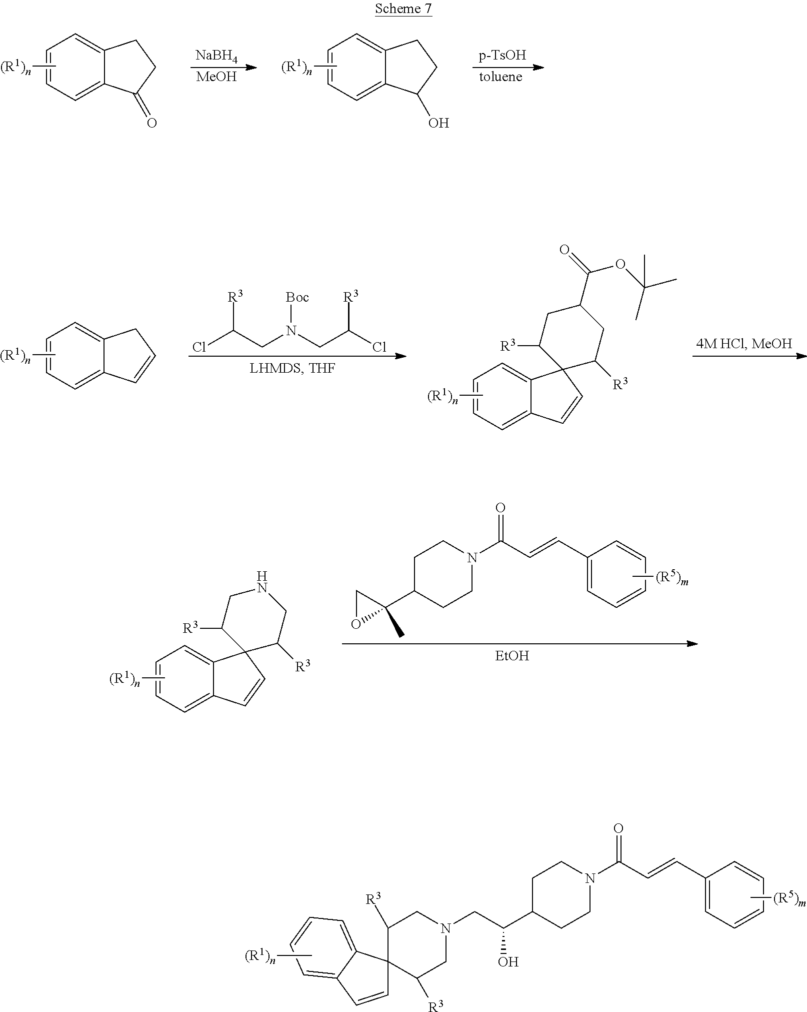

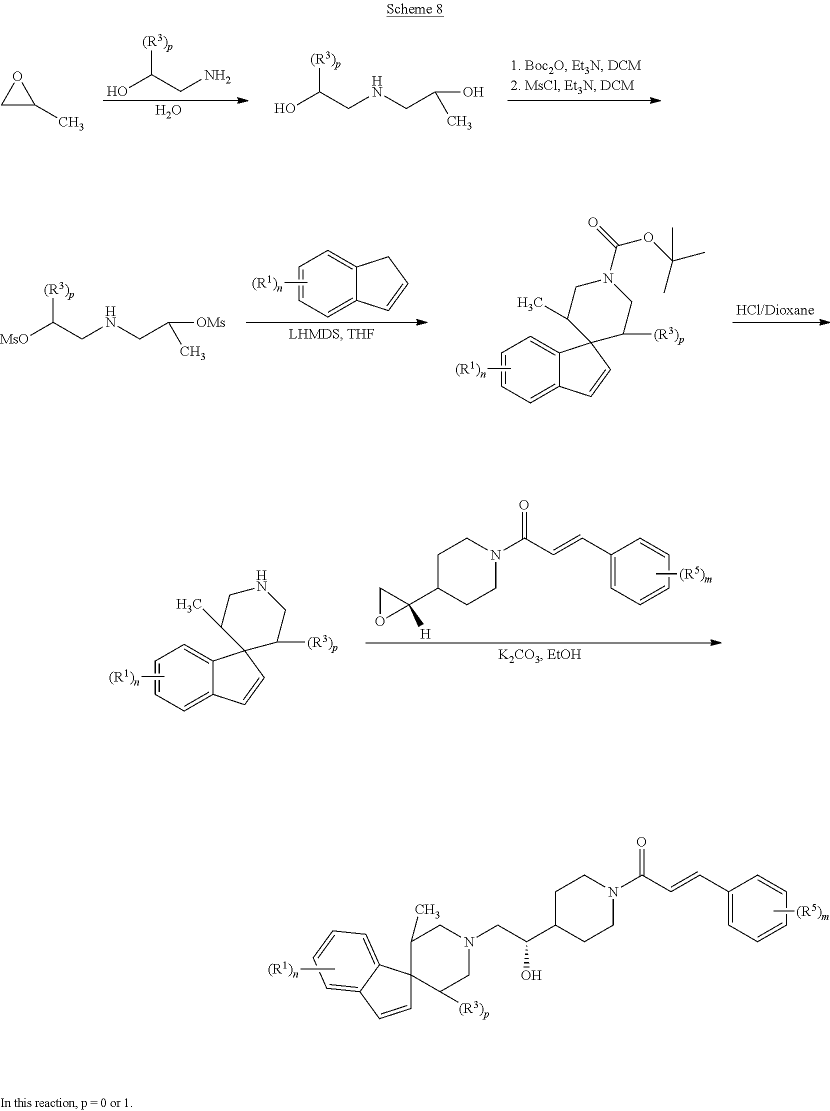

In the following scheme, the epoxidized intermediate, phenylmethyl-4-hydroxy-4-(2-oxiranyl)-1-piperidine carboxylate, may first be resolved into individual enantiomers using either (R,R)-Co(Salen) or (S,S)-Co(Salen) prior to nucleophilic ring opening

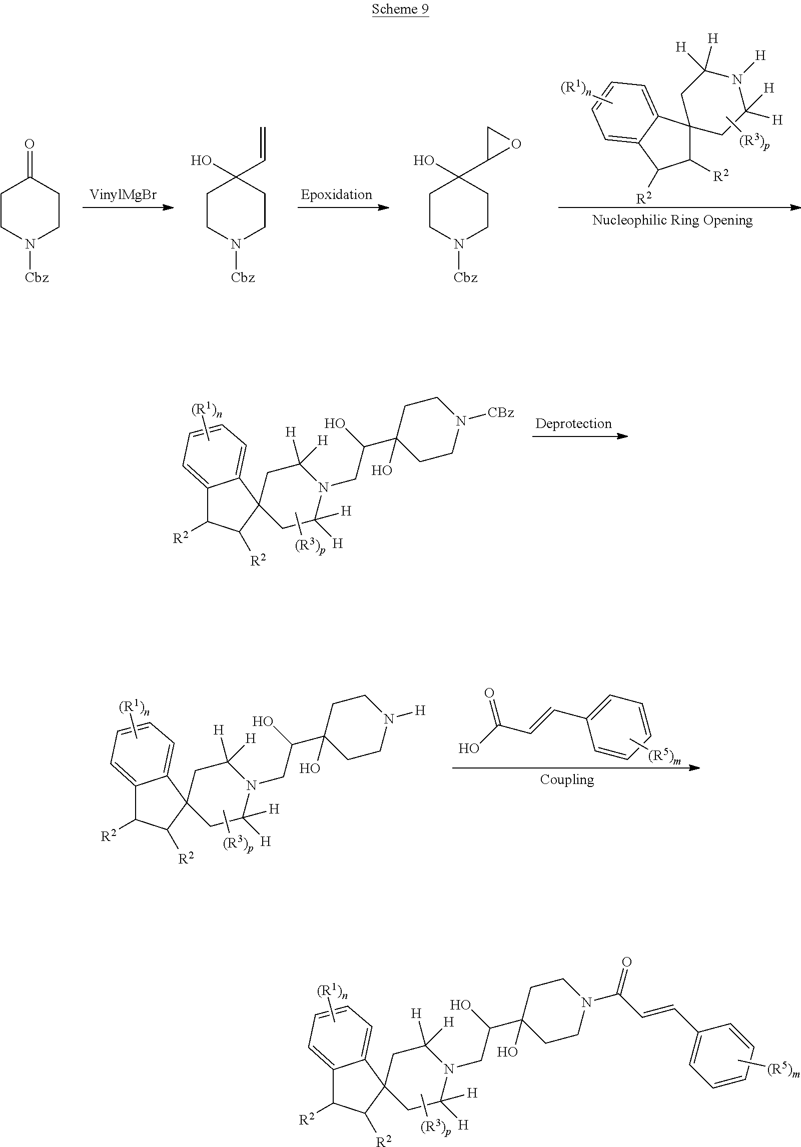

### EXAMPLES

The following examples are for illustrative purposes only and are not intended to limit the scope of the invention.

Mass spectra were obtained using either a Waters ZQ mass spectrometer or Micromass Platform 2 mass spectrometer and use electro-spray ionization to observe either MH+ or M−. Proton Nuclear Magnetic Resonance (¹H-NMR) spectra were recorded at 400 MHz, chemical shifts are reported in ppm downfield from Me₄Si, used as internal standard, and are assigned as singlets (s), doublets (d), doublets of doublets (dd), triplets (t), doublet of triplets (dt), quartets (q) multiplets (m) or are otherwise described in full. The prefix “br” refers to a broad peak; for example, a broad singlet may appear as br.s (or br s).

### Intermediate 1: {1-[(2E)-3-(3,5-difluorophenyl)-2-propenoyl]-4-piperidinyl}methanol

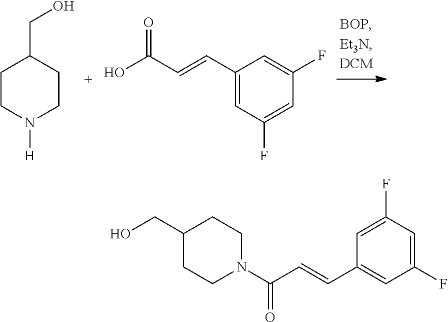

4-Piperidinemethanol (17.7 g, 154 mmol), 3,5-difluorocinnamic acid (28.3 g, 154 mmol) and benzotriazol-1-yloxytris(dimethylamino)phosphonium hexafluorophosphate (BOP) (78.2 g, 178 mmol) were dissolved in 700 mL dichloromethane (DCM). Triethylamine (46.6 g, 461 mmol) was added and the resulting solution was stirred at room temperature overnight. LCMS showed 100% conversion. The reaction mixture was concentrated and purified via silica gel column eluting with 0-75% ethyl acetate in hexanes to afford the product as a white solid (35 g, 81%). MS (ES) m/e 282 [M+1-1]⁺.

### Intermediate 2: 1-[(2E)-3-(3,5-difluorophenyl)-2-propenoyl]-4-piperidinecarbaldehyde

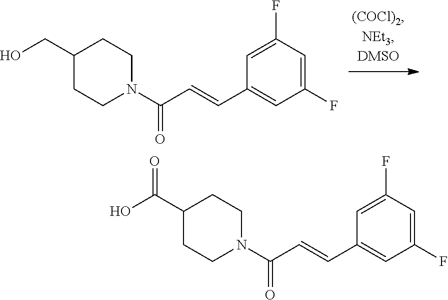

A 2-L round bottom flask was charged with 900 mL DCM and oxalylchloride (25.4 g, 200 mmol) and cooled to −78° C. Dimethylsulfoxide (DMSO) (31.2 g, 400 mmol) was added dropwise and the mixture was stirred at −78° C. for 10 mins. Then, {1-[(2E)-3-(3,5-difluorophenyl)-2-propenoyl]-4-piperidinyl}methanol (43.2 g, 150 mmol, dissolved in 100 mL of DCM and a few mL DMSO) was added slowly. After stirring for another 30 min at −78° C., NEt₃ (93.1 g, 920 mmol) was added slowly. The suspension was then stirred at −78° C. for 30 mins, then warmed to room temperature over 2 h. The mixture was diluted with 300 mL DCM and washed with 2×200 mL 2M HCl, 1×100 mL saturated NaHCO₃, dried over MgSO₄, and then filtered and concentrated to afford 1-[(2E)-3-(3,5-difluorophenyl)-2-propenoyl]-4-piperidinecarbaldehyde (35.4 g, 82%) as a brown oil. The material was used in the next step without further purification. MS (ES) m/e 280 [M+H]⁺. ¹H NMR (CDCl₃) δ(ppm): 9.72 (s, 1H), 7.56 (d, 1H), 7.04 (m, 2H), 6.90 (d, 1H), 6.82 (m, 1H), 4.40 (m, 1H), 4.00 (m, 1H), 3.36 (m, 1H), 3.22 (m, 1H), 2.56-2.64 (m, 1H), 2.02 (m, 2H), 1.67 (m, 2H).

### Intermediate 3: 1-[(2E)-3-(3,5-difluorophenyl)-2-propenoyl]-4-(2-oxiranyl)piperidine

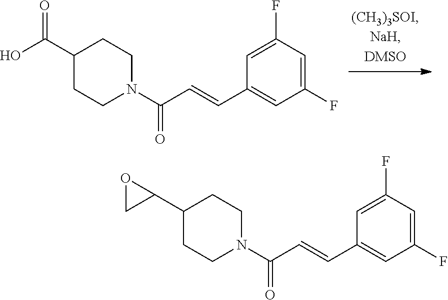

An oven dried 1000 mL flask was charged with (CH₃)₃SOI (46.1 g, 210 mmol) and 250 mL dry DMSO. The solution was then cooled to 0° C., whereupon 95% NaH (5.3 g, 210 mmol) was added in around 10 batches. The resulting mixture was stirred at 0° C. for 30 mins. The aldehyde (Intermediate 2, 45 g, 161 mmol) in 150 mL dry DMSO solution was added dropwise and the resulting solution was stirred at 0° C. for 30 mins. LCMS showed completed reaction. The reaction was then quenched with 800 mL water and poured into 1500 mL diethyl ether. The organic layer was separated and washed with 2×150 mL water and dried over MgSO₄ and concentrated. Crude LCMS showed >90% purity for the desired product in 58% yield as a light yellow oil which solidified to a yellow solid overnight. MS (ES) m/e 294 [M+H]⁺. ¹H NMR (CDCl₃) δ(ppm): 7.56 (d, 1H), 7.04 (dd, 2H), 6.91 (d, 1H), 6.80 (m, 1H), 4.74 (m, 1H), 4.11 (m, 1H), 3.05 (m, 1H), 2.77 (s, 2H), 2.61 (m, 1H), 1.95 (m, 1H), 1.70-1.80 (m, 1H), 1.30-1.60 (m, 4H).

### Intermediate 4: Phenylmethyl 4-(2-oxiranyl)-1-piperidinecarboxylate

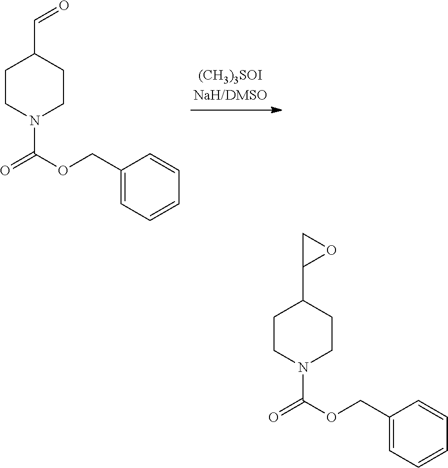

Trimethylsulfoxonium iodide (1.65 g, 7.5 mmol) was added in two portions to a solution of NaH (300 mg, 7.5 mmol) in anhydrous DMSO (10 mL) at room temperature. The resulting mixture was stirred for 1 h, whereupon a solution of phenylmethyl 4-formyl-1-piperidinecarboxylate (1.2 g, 5.0 mmol) in anhydrous DMSO (10 mL) was added. The reaction mixture was stirred at room temperature for 2 h, then poured into cold water (100 mL), and extracted with Et₂O (2×100 mL). The extracts were combined, washed with water, brine, and dried (Na₂SO₄). The solvent was removed in vacuo to give the title compound (0.95 g, 73%) as a colorless oil. MS (ES) m/e 262 [M+1-1]⁺.

### Intermediate 5: 1-[(2E)-3-(3,5-difluorophenyl)-2-propenoyl]-4-(2S-oxiranyl)piperidine

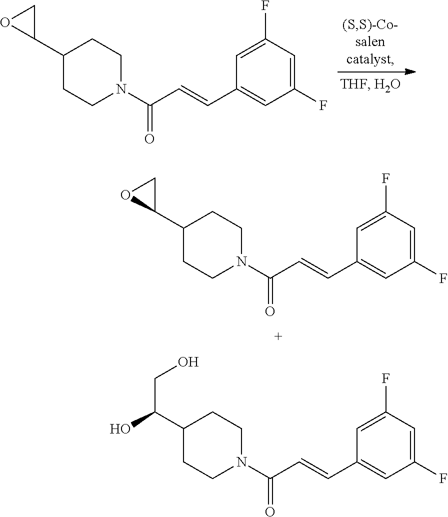

(S,S)-Co-salen catalyst (206 mg, 0.3 mmol) ((S,S)-(+)-N,N′-Bis(3,5-di-tert-butylsalicylidene)-1,2-cyclohexanediamino cobalt (II)) was dissolved in toluene (2 mL) in an open air flask. Glacial acetic acid (39 uL) was added and the reaction stirred at room temperature for 1 h. The reaction was then concentrated to a brown solid which was placed under high vacuum overnight. 1-[(2E)-3-(3,5-difluorophenyl)-2-propenoyl]-4-(2-oxiranyl)piperidine (2 g, 7.7 mmol) was dissolved in THF (2 mL). The catalyst was dissolved in THF (0.5 mL) and added to the solution of epoxide in an open air flask. The mixture was cooled to 0° C. and H₂O (69 uL) was added dropwise over 5 min. The reaction was warmed to room temperature and allowed to stir for 16 h. The reaction was then concentrated and purified by flash chromatography on a 120 g silica gel column (0 to 70% EtOAc/hexanes over 60 min.) to yield a yellow oil (805 mg, 40% yield). MS (ES) m/e 294 [M+H]^{+ 1}H NMR (400 MHz, DMSO-d₆) d ppm 7.55 (d, J=16 Hz, 1H), 7.03 (m, 2H), 6.91 (m, 1H), 6.81 (m, 1H), 4.74 (m, 1H), 4.13 (m, 1H), 3.15 (m, 1H), 2.79 (m, 4H), 1.90 (m, 1H), 1.47 (m, 4H). A sample of 1-[(2E)-3-(3,5-difluorophenyl)-2-propenoyl]-4-(2S-oxiranyl)piperidine from the above reaction was checked on a Chiralpac AD column with a 100% methanol mobile phase (0.9 mL/min) and found to have a retention time of 8.3 min, when compared to a racemic mixture (retention time 8.1 and 8.3 min) and found to be 99% ee.

### Example 1

### 1-{1-[(2E)-3-(3,5-difluorophenyl)-2-propenoyl]-4-piperidinyl}-2-(1′H-spiro[indene-1,4′-piperidin]-1′-yl)ethanol

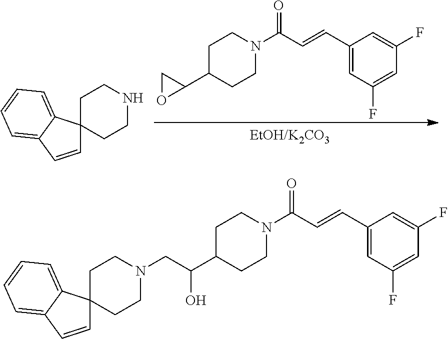

Spiro[indene-1,4′-piperidine]hydrochloride (110 mg, 0.50 mmol) was mixed with 1-[(2E)-3-(3,5-difluorophenyl)-2-propenoyl]-4-(2-oxiranyl)piperidine (147 mg, 0.50 mmol) and potassium carbonate (207 mg, 1.5 mmol) in 5 mL EtOH and heated to reflux for 3 h. After cooling to room temperature, the heterogeneous mixture was filtered. The filtrate was then concentrated, and the residue was purified by ISCO flash-chromatography to obtain the desired compound (136 mg, 57%). MS (ES) m/c 479 [M+H]⁺. ¹H NMR (400 MHz, CDCl₃) δ ppm 7.56 (d, J=15.31 Hz, 1H) 7.36 (dd, J=18.07, 6.78 Hz, 2H), 7.22-7.30 (m, 2H), 7.05 (dd, J=7.40, 1.13 Hz, 2H), 6.93 (d, J=15.31 Hz, 1H), 6.85 (t, J=5.27 Hz, 1H), 6.77-6.82 (m, 2H), 4.90-4.66 (m, 1H), 4.32-4.06 (m, 1H), 3.69-3.32 (m, 1H), 3.32-3.02 (m, 1H), 3.02-2.83 (m, 1H), 2.81-2.63 (m, 2H), 2.63-2.40 (m, 2H), 2.40-1.91 (m, 5H), 1.91-1.55 (m, 2H), 1.55-1.13 (m, 5H)

### Intermediate 6: 6-chloro-2,3-dihydro-1H-inden-1-ol

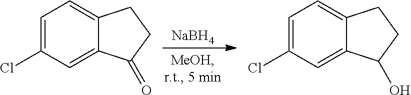

6-chloro-2,3-dihydro-1H-inden-1-one (10 g, 60 mmol) was dissolved in 300 mL MeOH and NaBH₄ (10.4 grams, 270 mmol) was added portionwise. After 5 min of stirring the reaction was evaporated, and water (150 mL) and DCM (200 mL) was added. The aqueous layer was extracted 1× with DCM (75 mL). The organic layers were combined, dried with sodium sulfate, and evaporated to yield 10 g of 6-chloro-2,3-dihydro-1H-inden-1-ol as a white solid. ¹H NMR (400 MHz, CDCl₃, δ): 7.39 (d, J=2.0 Hz, 1H), 7.24 (dd, J=2.0, 8.0 Hz, 1H), 7.18 (d, J=8.0 Hz, 1H), 5.21 (m, 1H), 3.02 (m, 1H), 2.79 (m, 1H), 2.52 (m, 1H), 2.07 (m, 1H), 1.96 (m, 1H).

### Intermediate 7: 5-chloro-1H-indene

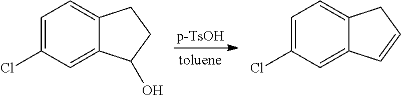

6-chloro-2,3-dihydro-1H-inden-1-ol (10 g, 59 mmol) was mixed with p-toluenesulfonic acid monohydrate (1.1 g, 5.9 mmol) in 250 mL toluene and refluxed 0.5 h with a Dean-Stark trap attached to the reaction flask. After cooling to room temperature, the reaction mixture was washed 1×10% aqueous sodium bicarbonate. The organic layer was dried over sodium sulfate, filtered, and evaporated to crude product. This material was loaded onto a silica column and eluted with 100% hexane to give 4.5 g of pure 5-chloro-1H-indene. ¹H NMR (400 MHz, CDCl₃, δ): 7.50 (d, J=2.0 Hz, 1H), 7.45 (d, J=8.0 Hz, 1H), 7.31 (d, J=2.0, 8.0 Hz, 1H), 6.93 (m, 1H), 6.72 (m, 1H), 3.45 (s, 2H).

### Intermediate 8: 1,1-dimethylethyl 5-chloro-1′H-spiro[indene-1,4′-piperidine]-1′-carboxylate

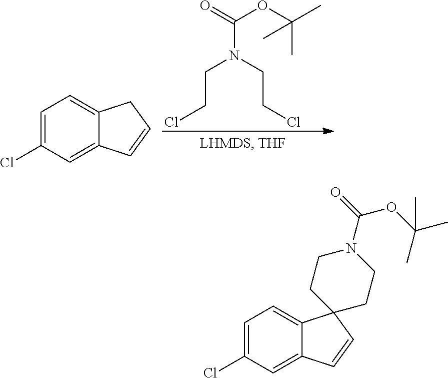

5-chloro-1H-indene (2 g, 13 mmol) dissolved in dry THF (10 mL) was added to a stirring solution of 1M lithium hexamethyldisilazide (LHMDS) (28 mL, 28 mmol) at 0° C. The mixture was stirred for 1 hr, and 1,1-dimethylethyl bis(2-chloroethyl)carbamate (3.2 g, 13 mmol) dissolved in THF (10 mL) was added dropwise. The resulting purple solution was taken out of the ice bath and allowed to stir overnight at room temperature. The mixture was diluted with water and DCM, and the layers were separated. The organic layer was dried over sodium sulfate, evaporated, then purified by flash column chromatography to yield 2 grams of a 50:50 mix of 5-Cl and 6-Cl regiomers. The regioisomers were separated by HPLC. Analysis by HMBC showed that the peak eluting second was the desired 5-Cl regioisomer. MS (ES) m/e 320 [M+H]⁺. ¹H NMR (400 MHz, CDCl₃, δ): 7.30 (d, J=1.76 Hz, 1H), 7.22 (d, J=8.03 Hz, 1H), 7.17 (dd, J=1.76 Hz, 8.03 Hz, 1H), 6.91 (d, J=5.64 Hz, 1H), 6.73 (d, J=5.64 Hz, 1H), 4.19 (m, 2H), 3.12 (m, 2H), 1.98 (m, 2H), 1.52 (s, 9H), 1.32 (m, 2H).

### Intermediate 9: 5-chlorospiro[indene-1,4′-piperidine]

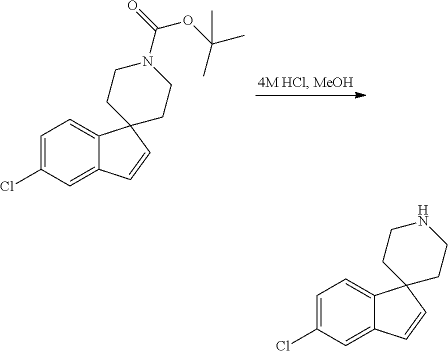

1,1-dimethylethyl 5-chloro-1′H-spiro[indene-1,4′-piperidine]-1′-carboxylate (1 g, 3.1 mmol) was mixed with 4M HCl in dioxane (15.7 mL, 63 mmol) and MeOH (40 mL) and stirred overnight. The reaction was then concentrated in vacuo, treated with DCM and 1 M NaOH. The layers were separated and the aqueous layer was back extracted 2× with DCM. The combined organic layers were dried over sodium sulfate and concentrated in vacuo to yield 5-chlorospiro[indene-1,4′-piperidine] which was used without further purification. MS (ES) m/c 220 [M+H]

### Example 2

### 2-(5-chloro-1′H-spiro[indene-1,4′-piperidin]-1′-yl)-1-{1-[(2E)-3-(3,5-difluorophenyl)-2-propenoyl]-4-piperidinyl}ethanol

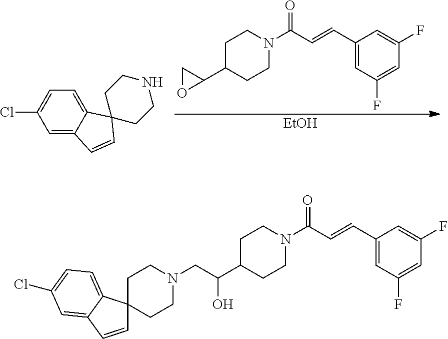

1-[(2E)-3-(3,5-difluorophenyl)-2-propenoyl]-4-[2-oxiranyl]piperidine (54 mg, 0.18 mmol) was mixed with 5-chlorospiro[indene-1,4′-piperidine] (45 mg, 0.2 mmol) in 2 mL EtOH and then heated to 160° C. in a microwave reactor for 10 min. The solvent was evaporated, the residue dissolved in 2 mL MeOH, and purified by HPLC to yield 70 mg of 2-(5-chloro-1′H-spiro[indene-1,4′-piperidin]-1′-yl)-1-{1-[(2E)-3-(3,5-difluorophenyl)-2-propenoyl]-4-piperidinyl}ethanol. MS (ES) m/e 513 [M+H]⁺. ¹H NMR (400 MHz, CDCl₃, δ): 7.56 (d, J=16.0 Hz, 1H), 7.32-7.26 (m, 2H), 7.19 (m, 1H), 7.04 (m, 2H), 6.96-6.87 (m, 2H), 6.81 (m, 1H), 6.71 (m, 1H), 4.80 (m, 1H), 4.15 (m, 1H), 3.83 (v. br. s., 1H), 3.55 (m, 1H), 3.22-3.06 (m, 2H), 2.89 (m, 1H), 2.70 (m, 2H), 2.62-2.39 (m, 2H), 2.31 (m, 1H), 2.24-1.95 (m, 3H), 1.85-1.59 (m, 2H), 1.38 (m, 4H).

### Intermediate 10: Phenylmethyl 4-[1-hydroxy-2-(1′H-spiro[indene-1,4′-piperidin]-1′-yl)ethyl]-1-piperidinecarboxylate

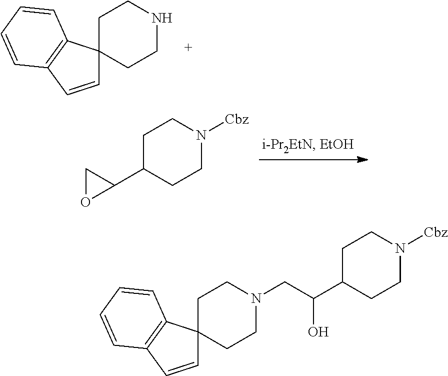

Spiro[indene-1,4′-piperidine]hydrochloride (500 mg, 2.2 mmol) was suspended in EtOH (11 mL) and i-Pr₂NEt (753 μL, 4.5 mmol) was added. Phenylmethyl 4-(2-oxiranyl)-1-piperidinecarboxylate (589 mg, 2.2 mmol) was then added and the reaction was warmed to 80° C. overnight. Additional phenylmethyl 4-(2-oxiranyl)-1-piperidinecarboxylate (290 mg, 1.1 mmol) was added and the reaction stirred an additional 4 h. The solvent was removed in vacuo and the crude material was purified via flash chromatography eluting with 0-10% MeOH in CH₂Cl₂ to give the title compound as a foamy yellow solid (436 mg, 44%). MS (ES) m/e 447 [M+H]⁺; ¹H NMR (400 MHz, CHLOROFORM-d) δ ppm 1.21-1.43 (m, 2H) 1.43-1.56 (m, 1H) 1.56-1.81 (m, 2H) 1.56-1.81 (m, 2H) 1.82-1.99 (m, 1H) 2.67-2.88 (m, 2H) 2.78 (d, 2H) 4.26 (br. s., 2H) 5.15 (s, 2H) 6.77-6.87 (m, 1H) 7.21-7.30 (m, 1H) 7.31-7.42 (m, 5H)

### Intermediate 11: 1-(4-piperidinyl)-2-(1′H-spiro[indene-1,4′-piperidin]-1′-yl)ethanol

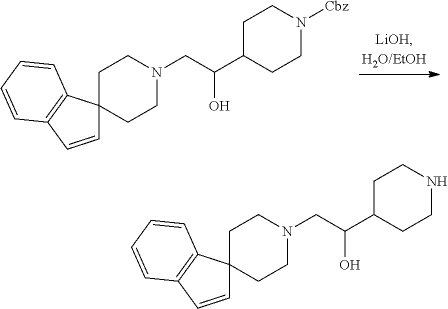

A solution of phenylmethyl 4-[1-hydroxy-2-(1′H-spiro[indene-1,4′-piperidin]-1′-yl)ethyl]-1-piperidinecarboxylate (436 mg, 1.0 mmol) and LiOH.H₂O (124 mg, 3 mmol) in EtOH:H₂O (4.9 mL, 3:1) was stirred at 90° C. overnight. The solvent was evaporated in vacuo and the resulting solid was extracted with CH₂Cl₂ several times. The solvent was removed and the crude material was used without purification. MS (ES) m/e 313 [M+H]⁺.

### Example 3

### N-(3-bromo-4-chlorophenyl)-4-[1-hydroxy-2-(1′H-spiro[indene-1,4′-piperidin]-1′-yl)ethyl]-1-piperidinecarboxamide

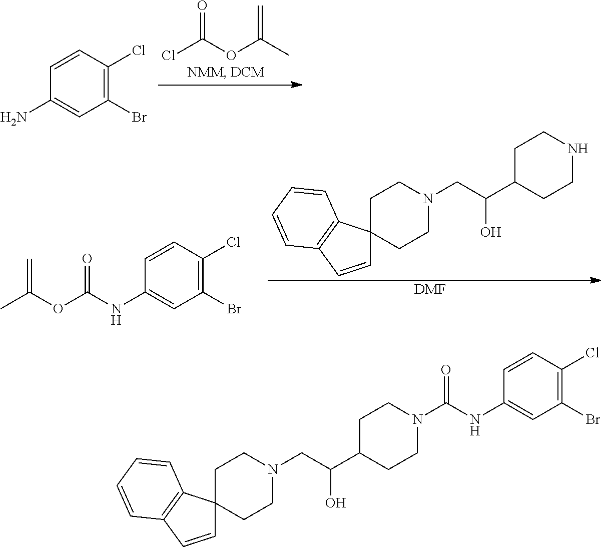

To a mixture of 3-bromo-4-chloroaniline (25 mg, 0.12 mmol) and N-methylmorpholine (13 μL, 0.12 mmol) in 1 mL of DCM was added isopropenyl chloroformate (13 μL, 0.12 mmol). The mixture was allowed to stir at room temperature for 2 h. The mixture was then washed with saturated aqueous sodium bicarbonate solution. The organic layer was separated and concentrated to dryness. The residue was dissolved in DMF (1 mL) and 1-(4-piperidinyl)-2-(1′H-spiro[indene-1,4′-piperidin]-1′-yl)ethanol (35 mg, 0.11 mmol) was added. The resulting solution was heated to 60° C. for 1 hr. The solution was then cooled to room temperature and purified by HPLC to obtain 9.2 mg (13%) of the desired product. MS (ES) m/e 544, 546 [M+H]⁺. ¹H NMR (500 MHz, DMSO-d₆) δ ppm 1.15-1.38 (m, 4H) 1.59 (d, J=10.74 Hz, 2H) 1.78 (d, J=13.67 Hz, 1H) 2.39 (td, J=14.04, 4.15 Hz, 1H) 2.48 (s, 1H) 2.57 (td, J=13.92, 3.91 Hz, 1H) 2.77 (t, J=12.70 Hz, 2H) 3.13-3.30 (m, 3H) 3.59 (d, J=12.21 Hz, 1H) 3.66 (d, J=11.72 Hz, 1H) 3.78 (br. s., 1H) 4.17 (d, J=11.72 Hz, 2H) 6.89 (d, J=5.86 Hz, 1H) 7.12 (d, J=5.37 Hz, 1H) 7.19-7.31 (m, 3H) 7.37 (t, 2H) 7.58 (d, J=9.28 Hz, 1H) 7.84 (d, J=2.44 Hz, 1H) 8.76 (s, 1H) 9.25 (br. s., 1H).

### Example 4

### 1-{1-[(2E)-3-(4-chlorophenyl)-2-propenoyl]-4-piperidinyl}-2-(1′H-spiro[indene-1,4′-piperidin]-1′-yl)ethanol

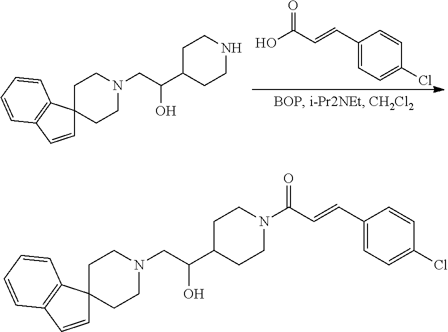

1-(4-piperidinyl)-2-(1′H-spiro[indene-1,4′-piperidin]-1′-yl)ethanol (30 mg, 0.1 mmol) was dissolved in CH₂Cl₂ (480 μL) and (2E)-3-(4-chlorophenyl)-2-propenoic acid (18 mg, 0.11 mmol) was added. i-Pr₂NEt (24 μL, 0.14 mmol) was then added followed by benzotriazol-1-yloxytris(dimethylamino)phosphonium hexafluorophosphate (BOP) (55 mg, 0.12 mmol). The reaction was stirred for 1 h and the reactions were diluted with CH₂Cl₂ and purified via preparatory HPLC to give the title compound as the corresponding trifluoroacetate salt (16 mg). MS (ES) m/e 477 [M+H]⁺; ¹H NMR (500 MHz, DMSO-d6) δ ppm 1.10-1.37 (m, 4H) 1.65 (br. s., 2H) 1.74-1.88 (m, 1H) 2.38 (d, J=3.91 Hz, 1H) 2.48 (s, 2H) 2.56 (d, J=4.39 Hz, 2H) 2.98-3.10 (m, 1H) 3.12-3.27 (m, 5H) 3.57 (d, J=12.70 Hz, 1H) 3.65 (d, J=12.70 Hz, 1H) 3.79 (br. s., 1H) 4.28-4.42 (m, 1H) 4.49-4.61 (m, 1H) 5.58 (br. s., 1H) 6.89 (d, J=5.37 Hz, 1H) 7.11 (d, J=5.86 Hz, 1H) 7.17-7.34 (m, 4H) 7.36 (d, J=6.84 Hz, 1H) 7.42-7.48 (m, 2H) 7.75 (d, J=8.79 Hz, 2H).

| Ex | | | | |
|---|---|---|---|---|
| # | Chemical Structure | Scheme | m/z | Name |
| 5 | 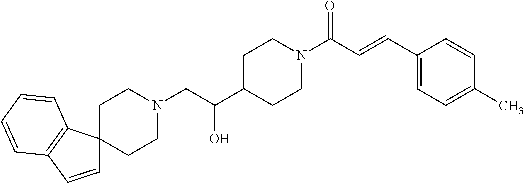 | 2 | 457 | 1-{1-[(2E)-3-(4- methylphenyl)-2-propenoyl]- 4-piperidinyl}-2-(1′H- spiro[indene-1,4′-piperidin]- 1′-yl)ethanol |
| 6 | 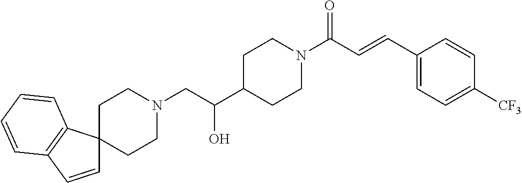 | 2 | 511 | 2-(1′H-spiro[indene-1,4′- piperidin]-1′-yl)-1-(1-{(2E)- 3-[4-(trifluoromethyl)phenyl]- 2-propenoyl}-4-piperidinyl) ethanol |
| 7 | 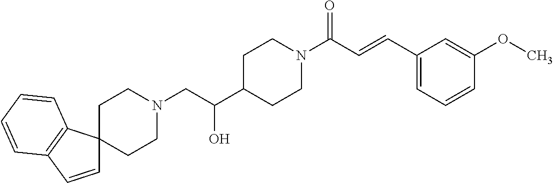 | 2 | 473 | 1-(1-{(2E)-3-[3-(methyloxy) phenyl]-2-propenoyl}-4- piperidinyl)-2-(1′H-spiro [indene-1,4′-piperidin]-1′- yl)ethanol |
| 8 | 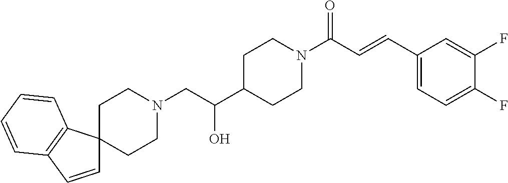 | 2 | 479 | 1-{1-[(2E)-3-(3,4-difluoro- phenyl)-2-propenoyl]-4- piperidinyl}-2-(1′H-spiro [indene-1,4′-piperidin]-1′- yl)ethanol |
| 9 | 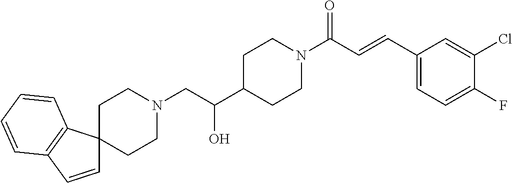 | 2 | 495 | 1-{1-[(2E)-3-(3-chloro-4- fluorophenyl)-2-propenoyl]- 4-piperidinyl}-2-(1′H- spiro[indene-1,4′-piperidin]- 1′-yl)ethanol |
| 10 | 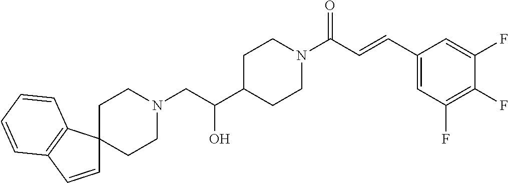 | 2 | 497 | 2-(1′H-spiro[indene-1,4′- piperidin]-1′-yl)-1-{1-[(2E)- 3-(3,4,5-trifluorophenyl)-2- propenoyl]-4-piperidinyl} ethanol |
| 11 | 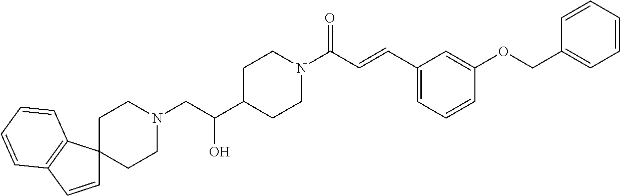 | 2 | 549 | 1-[1-((2E)-3-{3- [(phenylmethyl)oxy]phenyl}- 2-propenoyl)-4-piperidinyl]- 2-(1′H-spiro[indene-1,4′- piperidin]-1′-yl)ethanol |
| 12 | 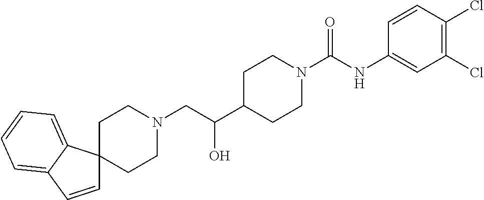 | 3 | 500 | N-(3,4-dichlorophenyl)-4-[1- hydroxy-2-(1′H-spiro[indene- 1,4′-piperidin]-1′-yl)ethyl]-1- piperidinecarboxamide |
| 13 | 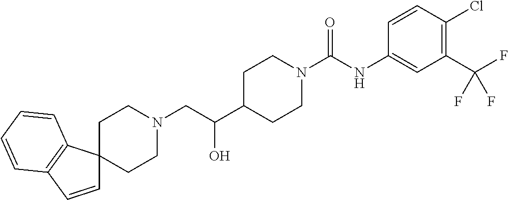 | 4 | 534 | N-[4-chloro-3- (trifluoromethyl)phenyl]-4-[1- hydroxy-2-(1′H-spiro[indene- 1,4′-piperidin]-1′-yl)ethyl]-1- piperidinecarboxamide |
| 14 | 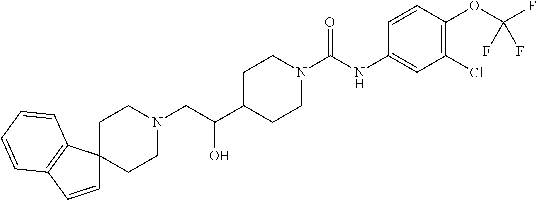 | 4 | 550 | N-{3-chloro-4- [(trifluoromethyl)oxy] phenyl}-4-[1-hydroxy-2- (1′H-spiro[indene-1,4′- piperidin]-1′-yl)ethyl]-1- piperidinecarboxamide |
| 15 | 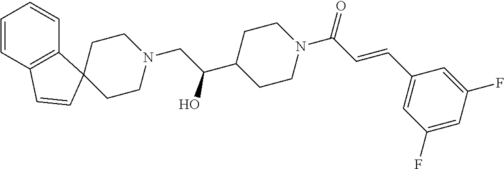 | 6 | 479 | (1R)-1-{1-[(2E)-3-(3,5- difluorophenyl)-2-propenoyl]- 4-piperidinyl}-2-(1′H- spiro[indene-1,4′-piperidin]- 1′-yl)ethanol |
| 16 | 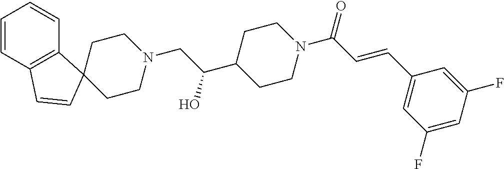 | 5 | 479 | (1S)-1-{1-[(2E)-3-(3,5- difluorophenyl)-2-propenoyl]- 4-piperidinyl}-2-(1′H- spiro[indene-1,4′-piperidin]- 1′-yl)ethanol |
| 17 | 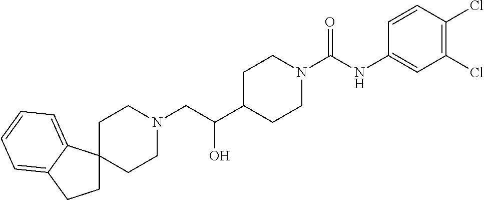 | 3 | 502 | N-(3,4-dichlorophenyl)-4-[2- (2,3-dihydro-1′H-spiro[indene- 1,4′-piperidin]-1′-yl)-1- hydroxyethyl]-1- piperidinecarboxamide |
| 18 | 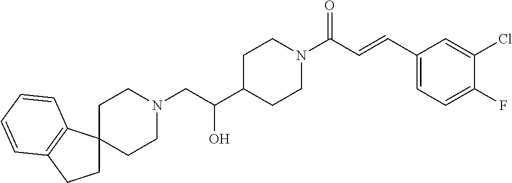 | 2 | 497 | 1-{1-[(2E)-3-(3-chloro-4- fluorophenyl)-2-propenoyl]-4- piperidinyl}-2-(2,3-dihydro- 1′H-spiro[indene-1,4′- piperidin]-1′-yl)ethanol |
| 19 | 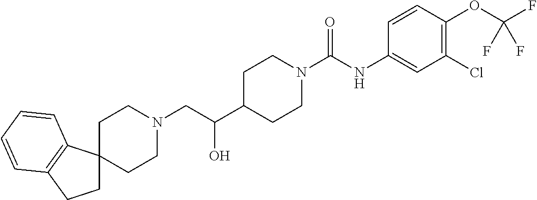 | 4 | 552 | N-{3-chloro-4- [(trifluoromethyl)oxy]phenyl}- 4-[2-(2,3-dihydro-1′H-spiro [indene-1,4′-piperidin]-1′-yl)- 1-hydroxyethyl]-1- piperidinecarboxamide |
| 20 | 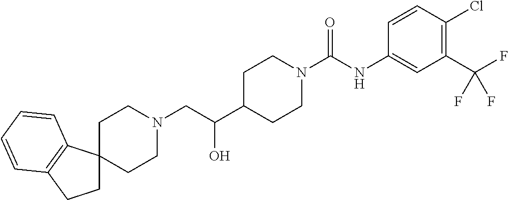 | 4 | 536 | N-[4-chloro-3- (trifluoromethyl)phenyl]-4- [2-(2,3-dihydro-1′H-spiro [indene-1,4′-piperidin]-1′- yl)-1-hydroxyethyl]-1- piperidinecarboxamide |
| 21 | 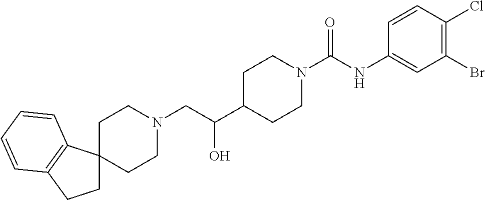 | 4 | 546 | N-(3-bromo-4-chlorophenyl)- 4-[2-(2,3-dihydro-l′H-spiro [indene-1,4′-piperidin]-1′- yl)-1-hydroxyethyl]-1- piperidinecarboxamide |
| 22 | 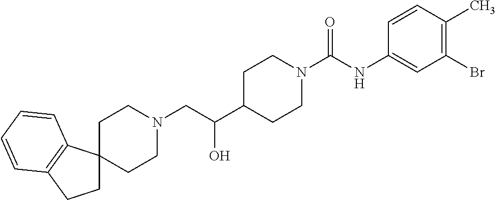 | 4 | 526 | N-(3-bromo-4-methylphenyl)- 4-[2-(2,3-dihydro-1′H-spiro [indene-1,4′-piperidin]-1′- yl)-1-hydroxyethyl]-1- piperidinecarboxamide |
| 23 | 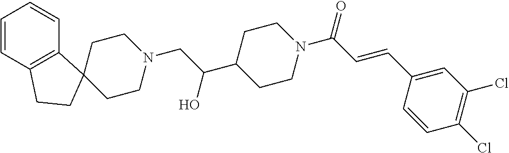 | 2 | 513 | 1-{1-[(2E)-3-(3,4-dichloro- phenyl)-2-propenoyl]-4- piperidinyl}-2-(2,3-dihydro- 1′H-spiro[indene-1,4′- piperidin]-1′-yl)ethanol |
| 24 | 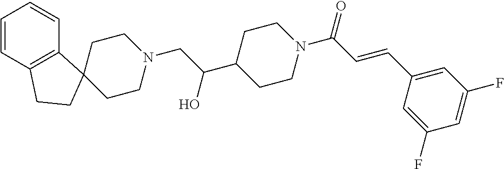 | 7 | 481 | 1-{1-[(2E)-3-(3,5-difluoro- phenyl)-2-propenoyl]-4- piperidinyl}-2-(2,3-dihydro- 1′H-spiro[indene-1,4′- piperidin]-1′-yl)ethanol trifluoroacetate (salt) |
| 25 | 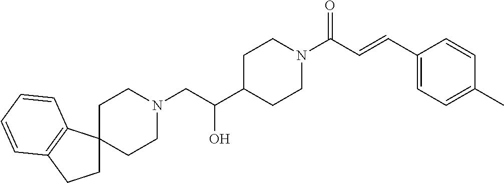 | 2 | 459 | 2-(2,3-dihydro-1′H-spiro [indene-1,4′-piperidin]-1′-yl)- 1-{1-[(2E)-3-(4- methylphenyl)-2-propenoyl]- 4-piperidinyl}ethanol |
| 26 | 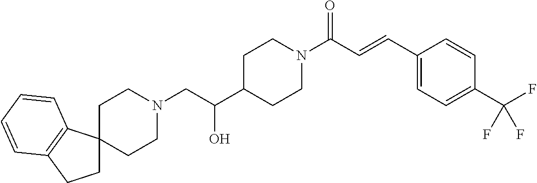 | 2 | 513 | 2-(2,3-dihydro-1′H-spiro [indene-1,4′-piperidin]-1′-yl)- 1-{1-[(2E)-3-(4- trifluoromethylphenyl)-2- propenoyl]-4-piperidinyl} ethanol |
| 27 | 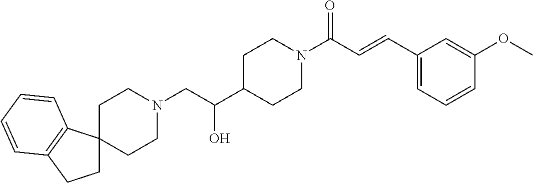 | 2 | 475 | 2-(2,3-dihydro-1′H-spiro [indene-1,4′-piperidin]-1′-yl)- 1-(1-{(2E)-3-[3- (methyloxy)phenyl]-2- propenoyl}-4-piperidinyl) ethanol |
| 28 | 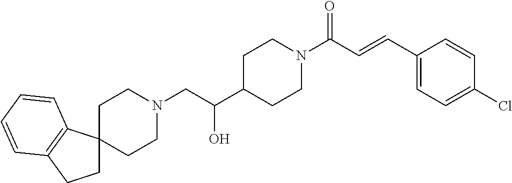 | 2 | 479 | 1-{1-[(2E)-3-(4- chlorophenyl)-2-propenoyl]- 4-piperidinyl}-2-(2,3-dihydro- 1′H-spiro[indene-1,4′- piperidin]-1′-yl)ethanol |
| 29 | 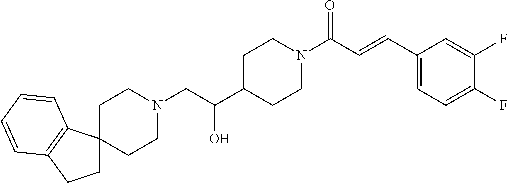 | 2 | 481 | 1-{1-[(2E)-3-(3,4- difluorophenyl)-2-propenoyl]- 4-piperidinyl}-2-(2,3-dihydro- 1′H-spiro[indene-1,4′- piperidin]-1′-yl)ethanol |
| 30 | 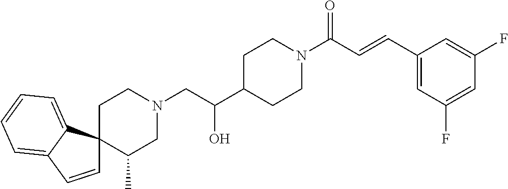 | 9 | 493 | 1-{1-[(2E)-3-(3,5- difluorophenyl)-2-propenoyl]- 4-piperidinyl}-2-[(1R,3′R)- 3′-methyl-1′H-spiro[indene- 1,4′-piperidin]-1′-yl]ethanol |
| 31 | 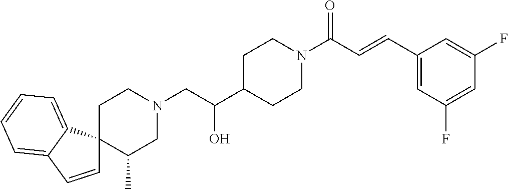 | 9 | 493 | 1-{1-[(2E)-3-(3,5- difluorophenyl)-2-propenoyl]- 4-piperidinyl}-2-[(1S,3′R)- 3′-methyl-1′H-spiro[indene- 1,4′-piperidin]-1′-yl]ethanol |
| 32 | 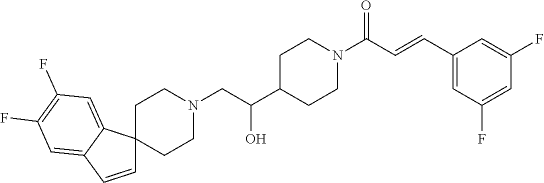 | 8 | 515 | 1-{1-[(2E)-3-(3,5- difluorophenyl)-2-propenoyl]- 4-piperidinyl}-2-(5,6- difluoro-1′H-spiro[indene- 1,4′-piperidin]-1′-yl)ethanol trifluoroacetate (salt) |
| 33 | 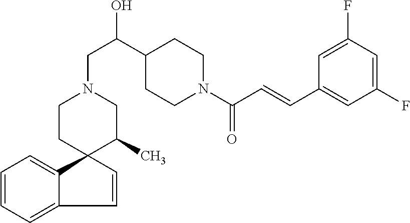 | 9 | 493 | 1-{1-[(2E)-3-(3,5- difluorophenyl)-2-propenyl]- 4-piperidinyl}-2-[(1S,3′S)- 3′-methyl-1′H-spiro[indene- 1,4′-piperidin]-1′-yl]ethanol |

1. A compound represented by the following structure: 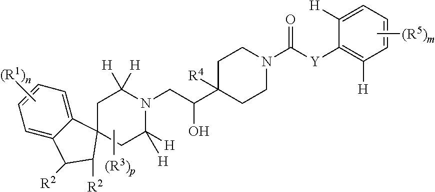
or a pharmaceutically acceptable salt thereof;
where each R¹ is independently halo, CF₃, C₁-C₄-alkyl, C₁-C₄-alkoxy, OCF₃, CN, C₁-C₆-alkyl-C(O)—NH—, C₁-C₆-alkyl-NH—C(O)—, —CH₂—N(R⁶)₂, —CH₂—O—R⁷, or heteroaryl;
each R² is H or, together with carbon atoms to which they are attached, form a double bond;
each R³ is each independently C₁-C₄-alkyl, hydroxy-C₁-C₄-alkyl, or C₁-C₄-alkoxy;
R⁴ is H, OH, F, CN, CF₃, or C₁-C₆-alkyl;
each R⁵ is independently halo, CF₃, C₁-C₄-alkyl, C₁-C₄-alkoxy, OCF₃, benzyloxy, or CN;
each R⁶ is independently H, C₁-C₄-alkyl, or, together with the nitrogen atom to which they are attached, form a 5- or 6-membered heterocycloalkyl group;
R⁷ is H, C₁-C₆-alkyl, benzyl, or phenyl;
Y is —NH— or 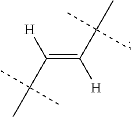
n is 0, 1, or 2;
m is 0, 1, 2 or 3; and
p is 0, 1, or 2.

2. The compound of claim 1, or a pharmaceutically acceptable salt thereof, wherein m is 1 or 2 and R⁵ is F, Cl, Br, —OCH₃, —CH₃, OCF₃, or O-benzyl.

3. The compound of either of claim 1, or a pharmaceutically acceptable salt thereof, wherein p is 0 or 1 and R³ is CH₃.

4. The compound of claim 1, or a pharmaceutically acceptable salt thereof, which compound is represented by the following structure: 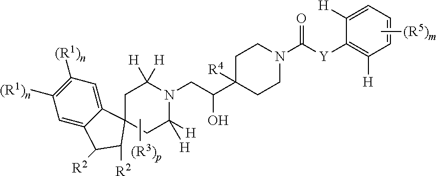
where each n is independently 0 or 1.

5. The compound of claim 1, or a pharmaceutically acceptable salt thereof, which compound is represented by the following structure: 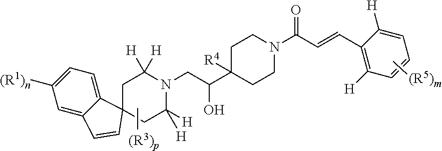
where n is 0 or 1; and R¹ is CH₃, F, or Cl.

6. The compound of claim 1, or a pharmaceutically acceptable salt thereof, wherein each R² is H; and Y is —NH—.

7. The compound of claim 1, or a pharmaceutically acceptable salt thereof, wherein each R² is H and Y is

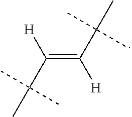

8. The compound of claim 1, or a pharmaceutically acceptable salt thereof, which compound is selected from the group consisting of:
1-{1-[(2E)-3-(3,5-difluorophenyl)-2-propenoyl]-4-piperidinyl}-2-(1′H-spiro[indene-1,4′-piperidin]-1′-yl)ethanol;
2-(5-chloro-1′H-spiro[indene-1,4′-piperidin]-1′-yl)-1-{1-[(2E)-3-(3,5-difluorophenyl)-2-propenoyl]-4-piperidinyl}ethanol;
1-{1-[(2E)-3-(3,5-difluorophenyl)-2-propenoyl]-4-piperidinyl}-2-[(1S,3′S)-3′-methyl-1′H-spiro[indene-1,4′-piperidin]-1′-yl]ethanol; and
1-{1-[(2E)-3-(3,5-difluorophenyl)-2-propenoyl]-4-piperidinyl}-2-[(1R,3′R)-3′-methyl-1′H-spiro[indene-1,4′-piperidin]-1′-yl]ethanol.

9. A composition that comprises a) the compound of claim 1, or a pharmaceutically acceptable salt thereof; and b) a pharmaceutically acceptable excipient.

10. A method of treating a disease comprising administering the composition of claim 9 or a pharmaceutically acceptable salt thereof to a patient in need thereof, wherein the disease is atherosclerosis, inflammatory pain, influenza, metabolic syndrome, multiple sclerosis, asthma, kidney disease, congestive heart failure, Alzheimer's disease, stroke, Crohn's disease, inflammatory bowel disease, endometriosis, or diabetes.
